(19) United States
(12) Patent Application Publication
(10) Pub. No.: US 2025/0064971 A1
(43) Pub. Date: Feb. 27, 2025

(54) SUSTAINED RELEASE FORMULATIONS AND METHODS OF USE THEREOF

(71) Applicant: VIRGINIA COMMONWEALTH UNIVERSITY, Richmond, VA (US)

(72) Inventors: Qingguo XU, Richmond, VA (US); Chaolong QIN, Richmond, VA (US); Tuo MENG, Richmond, VA (US); Aji Alex Moothedathu RAYNOLD, Richmond, VA (US); Matthew BANKS, Richmond, VA (US)

(21) Appl. No.: 18/727,821

(22) PCT Filed: Feb. 3, 2023
(86) PCT No.: PCT/US2023/012247

§ 371 Date: Jul. 10, 2024

Related U.S. Application Data
(60) Provisional application No. 63/306,535, filed on Feb. 4, 2022.

(51) Int. Cl. A61K 47/69; A61K 47/54

(52) U.S. Cl. CPC A61K 47/6927; A61K 47/541; A61K 47/542

(57) ABSTRACT

A method of delivering an active ingredient to a subject in need thereof is provided. The method includes administering to the subject a therapeutically effective amount of a formulation comprising biodegradable, polymeric microparticles and an active ingredient selected from the group consisting of levo-alpha-acetylmethadol (LAAM), nor-LAAM, and dinor-LAAM, wherein the active ingredient is part of a hydrophobic ion-pairing (HIP) complex and wherein the HIP complex is associated with the microparticles. Sustained release formulations containing an active ingredient are also provided.

### FIELD OF THE INVENTION

The invention is generally related to sustained release formulations containing levo-alpha-acetylmethadol (LAAM), nor-LAAM, or dinor-LAAM useful for the treatment of opioid use disorders.

### BACKGROUND OF THE INVENTION

Levo-alpha-acetylmethadol (LAAM), a derivative of methadone, has been approved by the FDA for use in therapy for opiate addiction. It has been found that LAAM can prevent opiate withdrawal symptoms and thus is used as a substitute for maintenance on methadone. LAAM is traditionally administered orally 3-4 times a week. LAAM is metabolized in the liver to produce two metabolites: nor-LAAM and dinor-LAAM. Each of the three compounds may provide for a therapeutic effect.

However, due to the frequency of dosing, patient compliance may suffer. Thus, new formulations that provide for a sustained release of LAAM and/or its metabolites are needed.

### SUMMARY

Described herein are formulations containing an active ingredient as part of a hydrophobic ion-pairing complex associated with biodegradable polymeric microparticles and methods of use thereof.

An aspect of the disclosure provides a formulation comprising biodegradable, polymeric microparticles and an active ingredient selected from the group consisting of levo-alpha-acetylmethadol (LAAM), nor-LAAM, and dinor-LAAM, wherein the active ingredient is part of a hydrophobic ion-pairing (HIP) complex and wherein the HIP complex is associated with the microparticles (e.g., encapsulated within or otherwise connected to the microparticles by ionic bonding or the like). In some embodiments, the HIP complex comprises an anion having a pKa ranging from −1.8 to 5.8. In some embodiments, the microparticles are poly(lactic acid/glycolic acid) (PLGA) microparticles. In some embodiments, the microparticles further comprise PLGA-polyethylene glycol (PEG) polymers. In some embodiments, the microparticles contain 15-25 wt % of PLGA-PEG polymers.

Another aspect of the disclosure provides a method of delivering an active ingredient to a subject in need thereof, comprising administering to the subject a therapeutically effective amount of a formulation as described herein. In some embodiments, the subject has opioid use disorder. In some embodiments, the formulation is administered subcutaneously or intramuscularly. In some embodiments, the formulation is administered less than once every two weeks, e.g., once in a time period of 1-3 months.

### BRIEF DESCRIPTION OF THE DRAWINGS

FIG. 1. Metabolization of LAAM into nor-LAAM and dinor-LAAM.

FIGS. 2A-C. SEM of control formulation 1 (A) and 2 (B); and (C) their drug release profiles.

FIGS. 3A-C. A schematic representation of the formation of Nor-LAAM-ion complex utilizing (A) oleic acid, (B) dioctylsulfosuccinic acid, and (C) pamoic acid.

FIG. 4. The drug release profiles of formulations 1A, 1, 2, and 3.

FIGS. 5A-B. (A) SEM of formulation 1B1, and (B) drug release profiles of formulations 1B1, 1B2, and 1B3.

FIGS. 6A-D. SEM of formulations (A) 1C1, (B) 1D1, and (C) 1E1, and (D) their drug release profiles.

FIG. 7. Simple linear regression analysis of drug loading (%) for norLAAM (circle) and pamoic acid (triangle) in HIP formulations of particle sizes from 5.3 to 20.7 μm made by emulsification. Positive relationship. 95% confident interval of the regression (dashed line).

FIGS. 8A-L. Release of HIP (pamoic acid). Cumulative drug release of formulations (A) F1B1, (B) F1B2, and (C) F1B3. Molar ratio of PAM:Nor-LAAM of formulations (D) F1B1, (E) F1B2, and (F) F1B3. Cumulative drug release of formulations (G) F1C1, (H) F1D1, and (I) F1E1. Molar ratio of PAM:Nor-LAAM of formulations (J) F1C1, (K) F1D1, and (L) F1E1.

### DETAILED DESCRIPTION

Embodiments of the disclosure provide sustained release formulations useful for the delivery of active agents to subjects in need thereof.

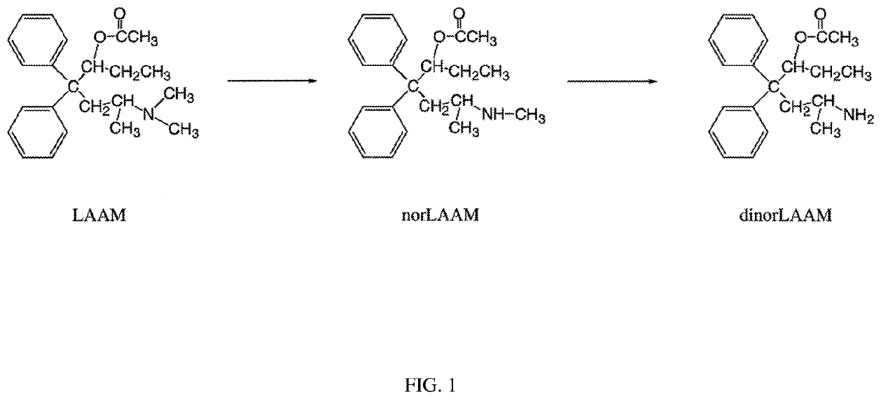

Levo-alpha-acetylmethadol (LAAM) is a derivative of methadone which is used to prevent opiate withdrawal symptoms. The clinical activity of LAAM is primarily based on the activity of two metabolites. LAAM is metabolized by the liver to produce the active demethylated metabolite nor-LAAM, which is further demethylated to a second active metabolite, dinor-LAAM (FIG. 1). The metabolites are more potent than the parent drug. In particular, nor-LAAM has a higher hERG inhibition IC₅₀ than LAAM. Exemplary embodiments provide formulations containing LAAM, nor-LAAM, or dinor-LAAM.

The active ingredient, e.g. nor-LAAM as demonstrated in the Example, may be provided as a hydrophobic ion-pairing complex by the addition of a counterion. Hydrophobic ion-pairing (HIP) is the interaction between a pair of oppositely charged ions held together by Coulombic attraction. HIP, as used herein, refers to the interaction between the active ingredient and its counterions, wherein the counterion is not H⁺ or HO⁻ ions. In some embodiments, the counterions are hydrophobic. In some embodiments, the counterions are provided by a hydrophobic acid or a salt of a hydrophobic acid. In some embodiments, the counterions are provided by bile acids or salts, fatty acids or salts, lipids, or amino acids. In some embodiments, the counterions are negatively charged (anionic). In some embodiments, the HIP complex comprises an anion having a pKa ranging from −1.8 to 5.8. Non-limiting examples of suitable counterions include oleic acid (sodium oleate), dioctyl sulfosuccinic acid (Sodium docusate), pamoic acid (disodium pamoate), 1-Hydroxy-2-naphthoic acid (xinafoic acid), 2-Naphthalene sulfonic acid (NSA), Cholesteryl hemisuccinate, Cholic acid (sodium cholate), Decanoic acid (sodium decanoate/sodium caprate), Dimyristoyl phosphatidyl glycerol (DMPG), Dioleoyl phosphatidic acid (DOPA), Docosahexaenoic acid, Linoleic acid, Sodium acetate, Sodium cholesteryl sulfate, Sodium deoxycholate, Sodium dodecyl benzenesulfonate (SDBS), Sodium dodecyl sulfate (sodium lauryl sulfate), Sodium laurate (sodium dodecanoate), Sodium stearate (stearic acid), Sodium taurodeoxycholate (STDC), Sodium tetradecyl sulfate, Sodium tripolyphosphate, Taurocholic acid (sodium taurocholate), Vitamin E (a-tocopherol) succinatepamoic acid, sodium sulfosuccinate (AOT), human serum albumin (HSA), dextran sulphate, sodium cholate, anionic lipids, amino acids, deoxycholic acid, pimelic acid, palmitic acid, or any combination thereof. In some embodiments, any suitable hydrophobic acid or a combination thereof may form a HIP pair with the active ingredient. In some embodiments, the hydrophobic acid may be a carboxylic acid (such as but not limited to a monocarboxylic acid, dicarboxylic acid, tricarboxylic acid), a sulfinic acid, a sulfenic acid, or a sulfonic acid. In some embodiments, a salt of a suitable hydrophobic acid or a combination thereof may be used to form a HIP pair with the active ingredient.

Without wishing to be bound by any theory, in some embodiments, HIP may increase the hydrophobicity and/or lipophilicity of the active ingredient. In some embodiments, increasing the hydrophobicity and/or lipophilicity of the active ingredient may be beneficial for particle formulations and may provide higher solubility of the active ingredient in organic solvents. Particle formulations that include HIP pairs may have improved formulation properties, such as drug loading and/or release profile. In some embodiments, slow release of the active ingredient from the particles may occur, due to a decrease in the active ingredient's solubility in aqueous solution. In addition, complexing the active ingredient with hydrophobic counterions may slow diffusion of the active ingredient within a polymeric matrix. In some embodiments, HIP occurs without covalent conjugation of the counterion to the active ingredient.

Further embodiments provide a microparticle comprising an active ingredient or HIP complex as described herein. The active ingredient or HIP complex may be loaded onto or into, encapsulated within, or otherwise associated with the particle. In particular embodiments, the microparticle comprises a biodegradable polymer or blends of polymers selected from the group consisting of poly(lactic-co-glycolic acid) (PLGA), poly(beta-amino ester) (PBAE), polycaprolactone (PCL), polyglycolic acid (PGA), polylactic acid (PLA), poly(acrylic acid) (PAA), poly-3-hydroxybutyrate (P3HB), poly(hydroxybutyrate-co-hydroxyvalerate), PLA/poly(D,L-lactide)-based polymers (e.g. RESOMER® R202H, R202S, R203H, R203S, and R205S), PLGA/poly(D,L-lactide-co-glycolide-based polymers (e.g. RESOMER® RG501H, RG502H, RG503H, RG504H, RG653H, RG752H, RG753H, and RG755S), poly(DL-lactide-co-glycolide) having a monomer DL-PLG ratio of 65:35 to 85:15, and PLA-PEG-PLA triblock copolymers. In some embodiments, the polymer may have a monomer LA: GA molar ratio 0:100 to 100:0, e.g. 50:50, 65:35, or 75:25. In some embodiments, the polymer is ester or carboxyl terminated. The polymers may have molecular weights ranging from, for example, 10-100 kDa, e.g. 18, 32, 45, 55, or 75 kDa. In some embodiments, the PLGA may have a molecular weight of about 5 kDa (PLGA-1A), 18 kDa (PLGA-2A), 34 kDa (PLGA-3A), 54 kDa (PLGA-4A), or 75 (PLGA-4E). Use of the biodegradable polymer microparticles can delay release of the active ingredient and permit sustained release of the active ingredient over some extended period of time.

In further embodiments, the microparticles may comprise PLGA-polyethylene glycol (PEG) polymers, PLA-PEG, or PCL-PEG diblock or triblock copolymers or mixtures thereof. The addition of a PLGA-PEG, PLA-PEG, or PCL-PEG can reduce significant inflammatory response and tissue reaction of PLGA, PLA, or PCL. The PEG can be any molecular weight, e.g. 500 Da to 40 kDa or higher and may be present in an amount of 1-50 wt %, e.g. 5-20 wt %. In some embodiments, the microparticles contain 1-100 wt % PLGA-PEG or PLA-PEG polymers, e.g. 15-25 wt % of PLGA-PEG or PLA-PEG polymers and 75-85 wt % PLGA polymers. In some embodiments, the PGLA to PGLA-PEG blend ratio (or PLA/PLA-PEG or PCL/PCL-PEG) is, for example, 4:1, 2:1, 1:1, 1:2, or 1:4.

The microparticles may have 0-100 wt % of any polymer, or combination of polymers, as described herein.

In some embodiments, the microparticles contain one or more emulsifiers (e.g., PLURONIC® emulsifiers, sugar esters, PVA, etc.)

In some embodiments, the microparticles are sized to be absorbed subcutaneously or intramuscularly. In some embodiments, the microparticles have a size of 5-30 μM. e.g. 10-25 μM.

In some embodiments, the microparticles may be formulated as liquid suspensions or as freeze-dried products. Suitable liquid preparations may include, but are not limited to, isotonic aqueous solutions, suspensions, emulsions, or viscous compositions that are buffered to a selected pH.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit. As used herein, the term “active ingredient” refers to any chemical and biological substance that has a physiological effect in human or in animals, when exposed to it.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. Other suitable excipients include, for example an inert diluent such as lactose; granulating and disintegrating agents such as cornstarch; binding agents such as starch; and lubricating agents such as magnesium stearate.

The compositions may be provided in the form of tablets, coated tablets, dragees, hard or soft gelatin capsules, solutions, emulsions or suspensions.

The compositions of the present disclosure may also contain other components such as, but not limited to, antioxidants, additives, adjuvants, buffers, tonicity agents, bioadhesive polymers, and preservatives. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values.

An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the bioactive agent, to adjust the osmolality of the composition, to adjust the viscosity of the composition, or for another reason, such as effecting drug delivery. Non-limiting examples of pharmaceutical additives of the present disclosure include sugars, such as, trehalose, mannose, D-galactose, and lactose.

In an embodiment, if a preservative is desired, the compositions may optionally be preserved with any well-known system such as benzyl alcohol with/without EDTA, benzalkonium chloride, chlorhexidine, Cosmocil® CQ, or Dowicil 200.

Further embodiments provide a method of delivering an active ingredient to a subject in need thereof comprising administering a formulation as described herein to the subject. The formulation may be used to treat any condition in which the active ingredient is useful for treating. In some embodiments, the subject may have an opioid abuse disorder or may be suffering from withdrawal symptoms. The formulation may reduce or prevent withdrawal symptoms including, but not limited to, apathy, irritability, recklessness, poor judgment, compulsion, aggression, anger, substance craving, mood disorders, and sleep disorders. In some embodiments, the formulation is useful for treating pain. In some embodiments, the formulation is used for pain relief in cancer patients.

A patient or subject to be treated by any of the compositions or methods of the present disclosure can mean either a human or a non-human animal including, but not limited to dogs, horses, cats, rabbits, gerbils, hamsters, rodents, birds, aquatic mammals, cattle, pigs, camelids, and other zoological animals.

In some embodiments, the peptide or composition is administered to the subject in a therapeutically effective amount. By a “therapeutically effective amount” is meant a sufficient amount to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000.0 mg per adult per day. In particular, the compositions contain 0.01, 0.05, 0.1, 0.5, 1.0, 2.5, 5.0, 10.0, 15.0, 25.0, 50.0, 100.0, 250.0 and 500.0 mg of the active ingredient for the symptomatic adjustment of the dosage to the subject to be treated. A medicament typically contains from about 0.01 mg to about 500.0 mg of the active ingredient, in particular from 1.0 mg to about 100.0 mg of the active ingredient. An effective amount of the drug is ordinarily supplied at a dosage level from 5 mg/kg to about 45 mg/kg of body weight per day, e.g. 10-40 mg/kg or 20-40 mg/kg.

“Sustained release” refers to release of a compound from a dosage form at a rate effective to achieve a therapeutic amount of the compound, or active metabolite thereof, in the systemic blood circulation over a prolonged period of time relative to that achieved by oral administration of an immediate formulation of the compound. In some embodiments, in vivo release of the compound occurs over a period of at least about 10, 15, 20, 25, or 30 days or more. In some embodiments, the formulation is administered only once in a time period of 2 weeks to 3 months, e.g. once within 2 weeks, once within a month, once within 6 weeks, once within 2 months, or once within three months. The formulations described herein may minimize the time required for administration of the active ingredient to the patient at each dose as well as may reduce the number of clinic visits for treatments that a patient needs to make, e.g. as compared to the LAAM oral solution product (ORLAAM®).

Any mode of administration may be used including, but not limited to, transdermal, intraperitoneal, intracranial, intracerebroventricular, intracerebral, intravaginal, intrauterine, oral, rectal, ophthalmic (including intravitreal or intracameral), nasal, topical (including buccal and sublingual), parenteral (including subcutaneous, intraperitoneal, intramuscular, intravenous, intradermal, intracranial, intratracheal, and epidural) administration.

Further embodiments provide a kit containing some or all of the components, reagents, supplies, and the like to practice a method according to the presently disclosed subject matter. In one embodiment, a kit comprises at least one container (e.g. a vial, tube, or ampoule) comprising a formulation as described herein.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example

### Control Formulations

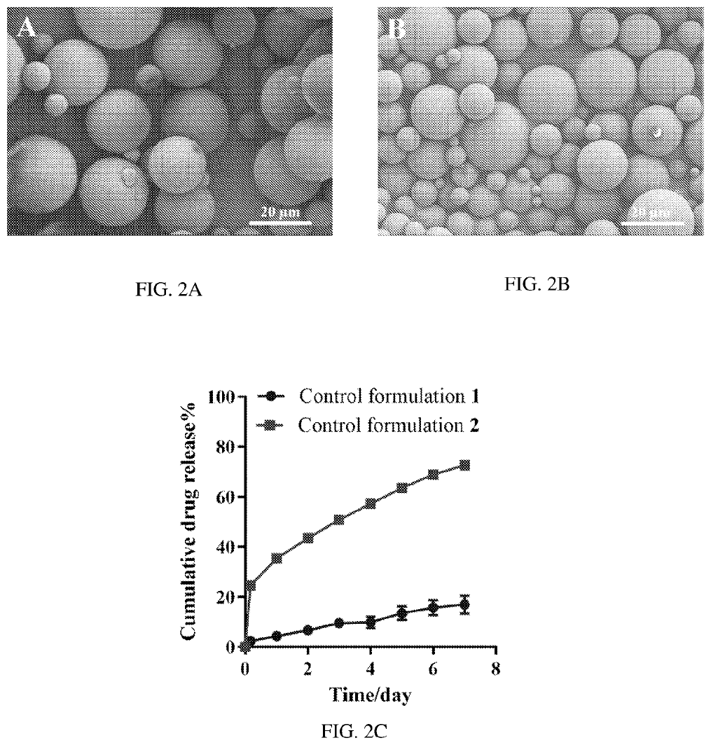

Control formulation 1 was made as plain microparticles (‘MPs’) without any counterions. MPs were prepared using PLGA-2A polymer (18 kDa). Nor-LAAM and PLGA polymer were fully dissolved in dichloromethane (DCM) to form solution, following adding to aqueous (1% Polyvinyl alcohol (PVA)) for homogenization (5,000 rpm). This control formulation results in microparticles with relatively low drug loading (3%) and inefficient release (see Table 1 and FIG. 2).

Control formulation 2 was prepared same as Control formulation 1 with slight modification. We added triethylamine (TEA) to remove same molar equivalent of HCl from Nor-LAAM hydrochloride salt form. Control formulation 2 improved drug loading from 3% to 13.2%, but high burst (>20%, at 4 hr) and fast release (>50%, at day 3) (see Table 1 and FIG. 2C).

TABLE 1

| | | | Drug | Drug | |
|---|---|---|---|---|---|
| | | | theoretical | actual | Size |
| Lot# | polymer | TEA | loading | loading | (μM) |
| Control | PLGA-2A | no | 20% | 3% | 20 |
| formulation 1 | | | | | |
| Control | PLGA-2A | yes | 20% | 13.2% | 23.8 |
| formulation 2 | | | | | |

### Hydrophobic Ion Pairing (HIP) Formulation

HIP formulations were made as MPs with different counterions. MPs were prepared using PLGA-1A, 2A, 3A, and 4A (E) (5, 18, 34, 54 kDa) with or without PLGA-PEG (50 kDa) polymers, respectively (Table 2). Nor-LAAM and pair ions were dissolved in water, separately, then were gradually mixed to precipitate hydrophobic complex. Nor-LAAM-ion complex and polymer were fully dissolved in dichloromethane (DCM) to form solution, following adding to aqueous (1% Polyvinyl alcohol (PVA)) for homogenization (5,000 rpm).

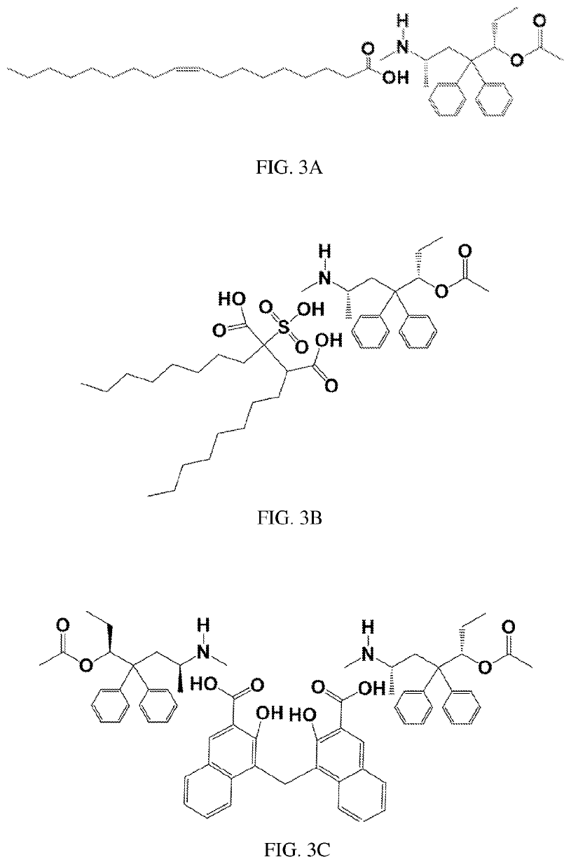

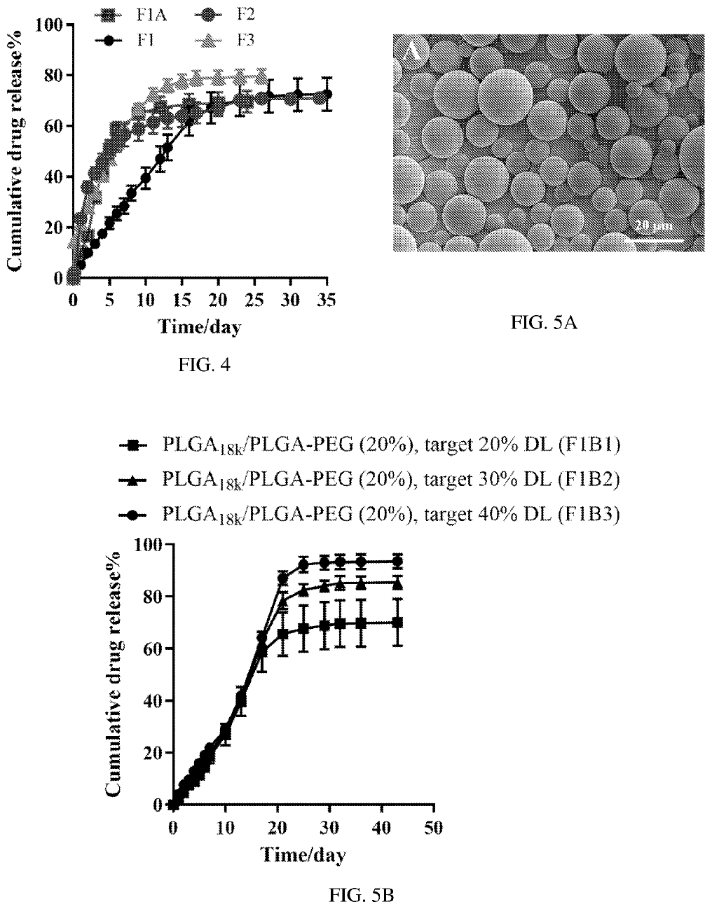

Oleic acid, dioctyl sulfosuccinic acid, and pamoic acid were used as counterions to form hydrophobic complex with Nor-LAAM. The formation of Nor-LAAM-ion complex was shown in FIG. 3. The HIP formulation 1 using pamoic acid as ion paring achieved prolonged release profile, compared to HIP formulations 2 and 3. HIP formulation 1A showed a fast release (>50%, at day 5), due to the low MW of PLGA-1A (5 kDa) (FIG. 4).

TABLE 2

| | | | Drug | Drug | |
|---|---|---|---|---|---|
| | | | theoretical | actual | Size |
| Lot# | polymer | counterion | loading | loading | (μM) |
| Formulation 1A | PLGA-1A | Pamoic acid | 20% | 11.2% | 17.4 |
| Formulation 1 | PLGA-2A | Pamoic acid | 20% | 13% | 25.1 |
| Formulation 2 | PLGA-2A | Oleic acid | 20% | 13.5% | 18.8 |
| Formulation 3 | PLGA-2A | Dioctyl | 20% | 12.8% | 20.4 |
| | | sulfosuccinic | | | |
| | | acid | | | |

### Increase the Drug Loading of Nor-LAAM in HIP Formulation Using Pamoic Acid

The HIP formulations were made as MPs using pamoic acid as pairing ion with Nor-LAAM. MPs were prepared using PLGA-2A polymer (18 kDa) blended with 20% PLGA-PEG (50 kDa) (Table 3). The target drug loading of Nor-LAAM were 20%, 30% and 40%, respectively. The MP preparation was the same as formulation 1 with slight modification (homogenization speed was increased to 7,000 rpm).

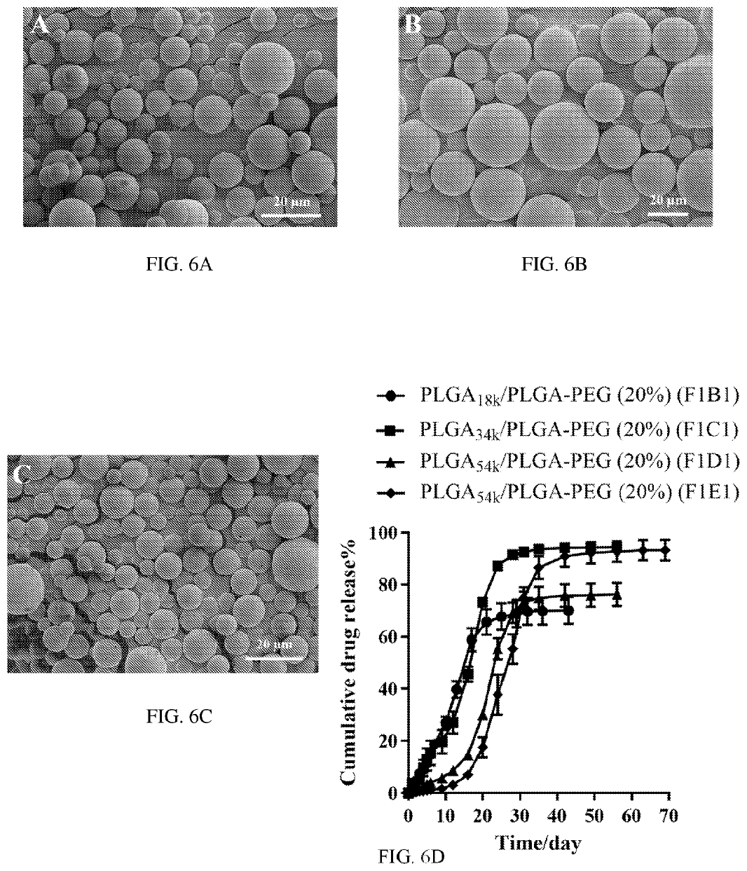

The HIP formulation 1 B1, B2, and B3 using pamoic acid as ion paring showed similar release profile before day 20. However, higher drug loading HIP formulation achieved more efficient drug release, compared to the lower drug loading formulation (F1B3 (18.2% DL)>F1B2 (14% DL)>F1B1 (10.1% DL)) after day 20 (FIG. 5B).

TABLE 3

| | | | Drug | Drug | |
|---|---|---|---|---|---|
| | | | theoretical | actual | Size |
| Lot# | polymer | counterion | loading | loading | (μM) |
| Formulation | PLGA-2A/PEG | Pamoic acid | 20% | 10.1% | 15.7 |
| 1B1 | (20%) | | | | |
| Formulation | PLGA-2A/PEG | Pamoic acid | 30% | 14% | 24.5 |
| 1B2 | (20%) | | | | |
| Formulation | PLGA-2A/PEG | Pamoic acid | 40% | 18.2% | 26.1 |
| 1B3 | (20%) | | | | |

### Prolong the Drug Release Profile of Nor-LAAM (Pamoic Acid) Using High MW PLGA Polymers

The HIP formulations were made as MPs using pamoic acid as pairing ion with Nor-LAAM. MPs were prepared using PLGA-3A and 4A (E) polymer (34 and 54 kDa) blended with 20% PLGA-PEG (50 kDa) (Table 4). The target drug loading of Nor-LAAM was 20%. The MP preparation was the same as formulation 1 with slight modification (homogenization speed was increased to 7,000 rpm).

TABLE 4

| | | | Drug | Drug | |
|---|---|---|---|---|---|
| | | | theoretical | actual | Size |
| Lot# | polymer | counterion | loading | loading | (μM) |
| Formulation | PLGA-3A/PEG | Pamoic acid | 20% | 12.2% | 21.8 |
| 1C1 | (20%) | | | | |
| Formulation | PLGA-4A/PEG | Pamoic acid | 20% | 12.5% | 27.3 |
| 1D1 | (20%) | | | | |
| Formulation | PLGA-4E/PEG | Pamoic acid | 20% | 12.3% | 26.1 |
| 1E1 | (20%) | | | | |

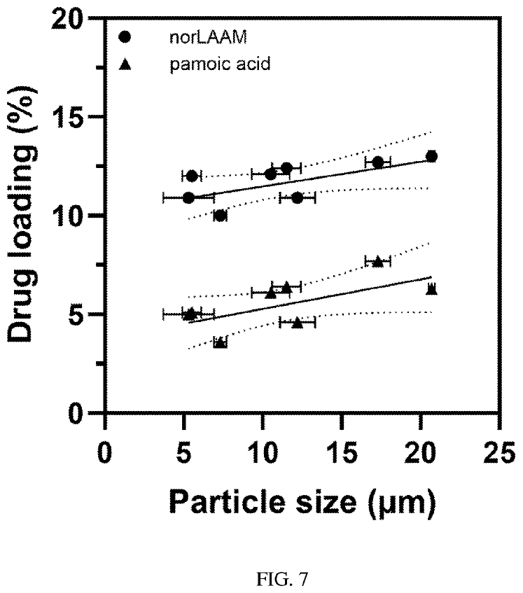

The HIP formulation 1D1 and 1E1 using pamoic acid as ion paring showed relative slow release of Nor-LAAM before day 10 and started to increase release rate around 3 weeks. (FIG. 6D).

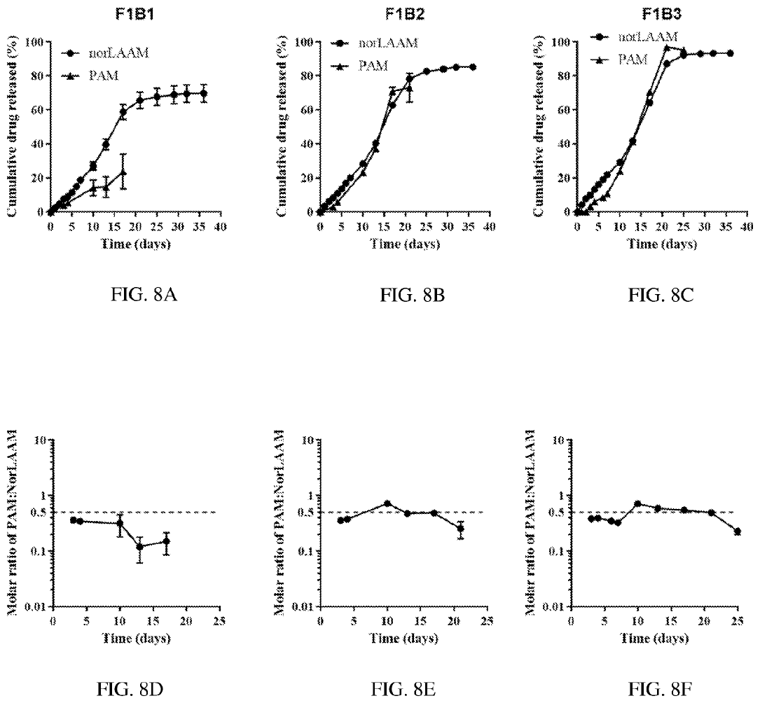

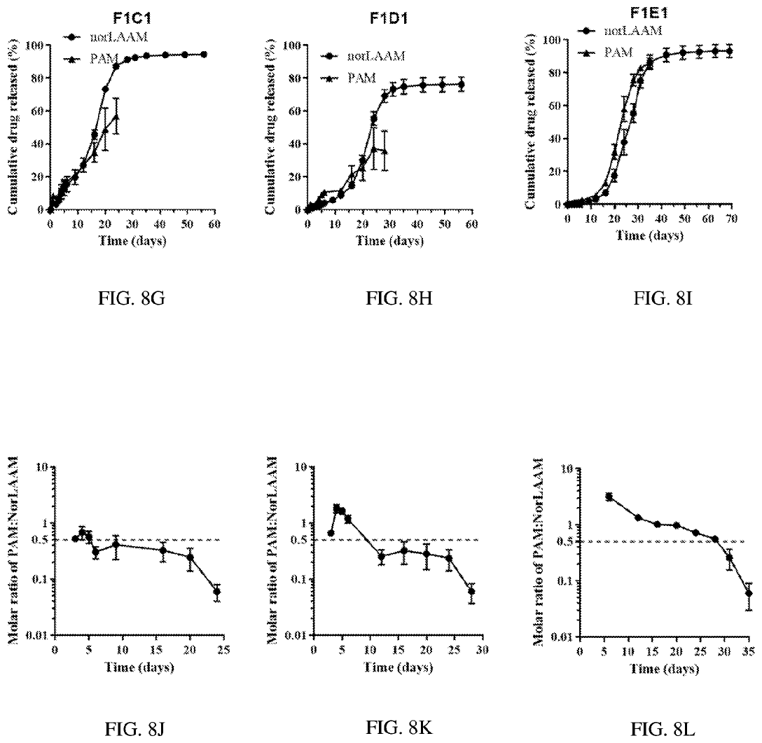

FIG. 7 is a simple linear regression analysis of drug loading (%) for norLAAM and pamoic acid in HIP formulations of particle sizes from 5.3 to 20.7 μm made by emulsification. The release of HIP (pamoic acid) over time was also monitored (FIGS. 8A-L).

In conclusion, the release of Nor-LAAM from MPs was substantially slower when formulated as a hydrophobic ion pair compared to the control formulations.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of delivering an active ingredient to a subject in need thereof, comprising administering to the subject a therapeutically effective amount of a formulation, comprising:
biodegradable, polymeric microparticles; and
an active ingredient selected from the group consisting of levo-alpha-acetylmethadol (LAAM), nor-LAAM, and dinor-LAAM, wherein the active ingredient is part of a hydrophobic ion-pairing (HIP) complex and wherein the HIP complex is associated with the microparticles.

2. The method of claim 1, wherein the subject has opioid use disorder.

3. The method of claim 1, wherein the formulation is administered subcutaneously or intramuscularly.

4. The method of claim 1, wherein the formulation is administered once in a time period of 1-12 months.

5. The method of claim 1, wherein the HIP complex comprises an anion having a pKa ranging from −1.8 to 5.8.

6. The method of claim 1, wherein the HIP complex contains pamoic acid.

7. The method of claim 1, wherein the biodegradable, polymeric microparticles are poly(lactic acid/glycolic acid) (PLGA) microparticles.

8. The method of claim 7, wherein the microparticles further comprise PLGA-polyethylene glycol (PEG) polymers.

9. The method of claim 8, wherein the microparticles contain 15-25 wt % of PLGA-PEG polymers.

10. The method of claim 1, wherein the HIP complex is encapsulated within the microparticles.

11. The method of claim 1, wherein the microparticles have a size of 10-30 μM.

12. A formulation, comprising:
biodegradable, polymeric microparticles; and
an active ingredient selected from the group consisting of levo-alpha-acetylmethadol (LAAM), nor-LAAM, and dinor-LAAM, wherein the active ingredient is part of a hydrophobic ion-pairing (HIP) complex and wherein the HIP complex is associated with the microparticles.

13. The formulation of claim 12, wherein the HIP complex comprises an anion having a pKa ranging from −1.8 to 5.8.

14. The formulation of claim 12, wherein the HIP complex contains pamoic acid.

15. The formulation of claim 12, wherein the biodegradable, polymeric microparticles are poly(lactic acid/glycolic acid) (PLGA) microparticles.

16. The formulation of claim 15, wherein the microparticles further comprise PLGA-polyethylene glycol (PEG) polymers.

17. The formulation of claim 16, wherein the microparticles contain 15-25 wt % of PLGA-PEG polymers.

18. The formulation of claim 12, wherein the HIP complex is encapsulated within the microparticles.

19. The formulation of claim 12, wherein the microparticles have a size of 10-30 μM.
